(19) United States
(12) Patent Application Publication
(10) Pub. No.: US 2007/0167711 A1
(43) Pub. Date: Jul. 19, 2007

(54) IMAGE DIAGNOSTIC APPARATUS

(76) Inventor: Masashi Seki, Tokyo (JP)

Correspondence Address: Patrick W. Rasche;Armstrong Teasdale LLP, Suite 2600, One Metropolitan Square, St. Louis, MO, 63102, US

(21) Appl. No.: 11/561,106

(22) Filed: Nov. 17, 2006

(30) Foreign Application Priority Data
Nov. 24, 2005 (JP) 2005-339017

(51) Int. Cl. A61B 5/05; A61B 8/00

(52) U.S. Cl. 600/407; 600/443

(57) ABSTRACT

In this invention, diagnosis efficiency can be improved. The first slice image is displayed through transmission corresponding to the transmitting degree preset by the first transmitting degree setting unit and the second slice image is displayed through transmission corresponding to the transmitting degree preset by the second transmitting degree setting unit. Moreover, the second slice image is convoluted on the first slice image corresponding to the position preset by the convolution position setting unit. For example, the first slice image of the affected area of the subject generated in the past and the newly generated second slice image of the same affective area of the subject are convoluted with each other and displayed through transmission.

### CROSS REFERENCE TO RELATED APPLICATIONS

This application claims the benefit of Japanese Application No. 2005-339017 filed Nov. 24, 2005.

### BACKGROUND OF THE INVENTION

The present invention relates to an image diagnostic apparatus and particularly to an image diagnostic apparatus for displaying a plurality of images of a subject.

On the occasion of implementing image diagnosis with an image diagnostic apparatus such as an ultrasonic diagnostic apparatus, images of the subject are displayed on the display screen.

For example, an image diagnostic apparatus displays a slice image of a slicing plane of a subject on a display screen of a display unit. Moreover, a characteristic image of subject showing characteristics of the subject such as name, sex, and diagnostic report is displayed on the display screen.

More concretely, an ultrasonic diagnostic apparatus in the image diagnostic apparatus generates, for example, slice images of slicing planes of a subject and displays these slice images on the display screen on the basis of an echo signal acquired by transmitting the ultrasonic wave to the subject and then implementing scanning to receive the ultrasonic wave reflected from the subject to which the ultrasonic wave has been transmitted. A characteristic image of the subject showing characteristic of the subject to which the scanning has been implemented is generated on the basis of the characteristic information inputted to the operating apparatus by an operator and is then displayed on the display screen. Particularly, the ultrasonic diagnostic apparatus is often used in the medical field such as in prenatal testing and heart examinations or the like, because a slice image of each slicing plane of the subject can be photographed easily on a real-time basis.

In this ultrasonic diagnostic apparatus, various display modes such as B mode (Brightness mode), M mode (Motion mode), Doppler mode or the like are provided. In the B mode, an image attained by converting variation in intensity of the ultrasonic wave echo reflected from the subject into variation in luminance is displayed. For example, the B mode is employed for imaging a slice image of the slicing plane of the subject. In the M mode, luminance of the part corresponding to one sound line of the ultrasonic wave echo is displayed on the time series basis in a plurality of B mode images displayed sequentially on the time series basis. For example, the M mode is employed for imaging movement of the heart including valve movement of the heart in the subject. Moreover, in the Doppler mode, the Doppler effect is employed, in which frequency of the ultrasonic wave echo reflected by a moving body is shifted in proportion to the moving velocity of the moving body. For example, the Doppler mode is employed for imaging blood flowing information such as flowing velocity of the blood flowing in the subject (for example, refer to the patent document 1).

[Patent Document 1] Japanese Unexamined Patent Publication No. 2002-112254

In the image diagnostic apparatus such as the ultrasonic diagnostic apparatus explained above, a plurality of images are displayed in parallel on the display screen when a plurality of images of the subject are compared with each other. Here, a plurality of images are arranged in each display area of the display screen.

For example, slice images of an affected area of the subject picked up in the past and slice images of the same affected area of the subject newly picked up are respectively arranged in parallel on the display screen. Accordingly, changes on the time axis of such affected area of the subject can be detected. Moreover, for example, slice images picked up for the photographing region corresponding to the affected area in the subject and slice images picked up for the photographing region corresponding to such affected area are respectively arranged in parallel on the display screen. Accordingly, conditions of the affected area can be detected.

However, in the case where a plurality of images are compared with each other, the portions to be compared of the respective images are displayed separately on the display screen in order to display in parallel a plurality of images on the display screen as explained above.

Therefore, it has been difficult in some cases to detect the portions to be compared and it has also been difficult to quickly implement diagnosis. Accordingly, in some cases, it has been difficult to realize higher diagnosis efficiency.

### SUMMARY OF THE INVENTION

Therefore, an object of the present invention is to provide an image diagnostic apparatus which can improve diagnosis efficiency.

In order achieve the object explained above, the image diagnostic apparatus of the present invention relates to an image diagnostic apparatus including a display unit for displaying a second image of a subject on a first image of the same subject through convolution, comprising a first transmitting degree setting unit for setting a transmitting degree to display the first image on the display unit through transmission and a second transmitting degree setting unit for setting a transmitting degree to display the second image on the display unit through transmission, wherein the display unit displays the first image through transmission corresponding to the transmitting degree preset by the first transmitting degree setting unit and also displays the second image through transmission corresponding to the transmitting degree preset by the second transmitting degree setting unit.

According to the present invention, an image diagnostic apparatus for improving diagnosis efficiency can be provided.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram illustrating a structure of an ultrasonic diagnostic apparatus 1 as an embodiment of the present invention.

FIG. 2 is a flowchart showing operations of the ultrasonic diagnostic apparatus 1 according to the first embodiment of the present invention.

FIGS. 3a and 3b are diagrams illustrating profiles of operations of the ultrasonic diagnostic apparatus 1 according to the first embodiment of the present invention.

FIG. 4 is a flowchart showing operations of the ultrasonic diagnostic apparatus 1 according to the second embodiment of the present invention.

FIGS. 5a and 5b are diagrams illustrating profiles of operations of ultrasonic diagnostic apparatus 1 according to the second embodiment of the present invention.

FIG. 6 is a block diagram illustrating a structure of an ultrasonic diagnostic apparatus 1a according to the third embodiment of the present invention.

FIG. 7 is a diagram illustrating images displayed with a display unit 41 in the third embodiment of the present invention.

### DETAILED DESCRIPTION OF THE INVENTION

The embodiments of the present invention will be explained below.

### First Embodiment

The first embodiment of the present invention will be explained with reference to the accompanying drawings.

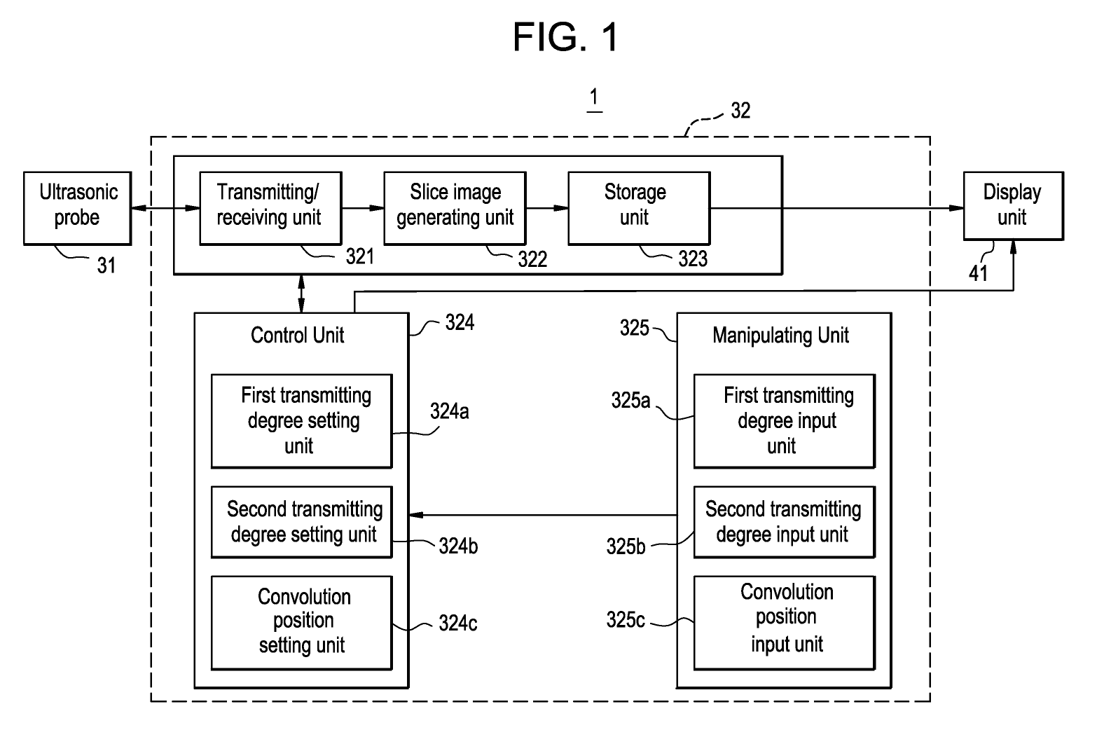

FIG. 1 is a block diagram illustrating a structure of an ultrasonic diagnostic apparatus according to the first embodiment of the present invention.

As illustrated in FIG. 1, the ultrasonic diagnostic apparatus 1 of this embodiment comprises an ultrasonic probe 31, an operating console 32, and a display unit 41.

The ultrasonic diagnostic apparatus 1 of the present embodiment generates and displays slice images of a subject on the basis of an echo signal obtained by transmitting the ultrasonic wave to the subject and then implementing scanning to receive the ultrasonic wave reflected from the subject to which the ultrasonic wave has been transmitted. Here, a plurality of slice images of the subject are convoluted with each other and are then displayed on the display screen.

More concretely, the second slice image as the new slice image of the affected area of a subject is convoluted to the first slice image as the slice image in the past of the affected area of the same subject and are then displayed on the display screen.

Each unit will be explained below sequentially.

The ultrasonic probe 31 includes a plurality of ultrasonic wave vibrators (not illustrated) and such ultrasonic wave vibrators, for example, are equally arranged in the shape of matrix. The ultrasonic wave vibrators in the ultrasonic probe 31 are constituted to include, for example, piezoelectric materials such as PZT (lead titanate zirconate) ceramics to convert electric signal to sound wave for transmission and to convert sound wave received to electric signal. The ultrasonic probe 31 acquires the raw data by implementing scanning of a subject. Details will be explained later but the ultrasonic probe 31 is used with the surface where the ultrasonic wave vibrators are provided placed in contact with the surface of the subject. Moreover, the ultrasonic probe 31 acquires echo signal by transmitting the ultrasonic wave to the subject from the ultrasonic wave vibrators corresponding to the drive signal from a transmitting/receiving unit 32 on the basis of a control signal outputted from a control unit 324 in the operating console 32 and then implementing scanning to receive, with the ultrasonic wave vibrators, the ultrasonic wave reflected from the subject to which the ultrasonic wave has been transmitted. This echo signal is then outputted to the transmitting/receiving unit 321 as the raw data.

In this embodiment, the ultrasonic probe 31 acquires the first echo signal by implementing scanning of the subject at the first time and also acquires the second echo signal by implementing the scanning of the subject at the second time different from the first time. For example, the second echo signal is acquired by implementing scanning of the affected area in the subject after the first echo signal is acquired by implementing scanning of the same affected area of the subject in the past.

The operating console 32 comprises, as illustrated in FIG. 1, a transmitting/receiving unit 321, a slice image generating unit 322, a storage unit 323, a control unit 324, and an operating unit 325. Each unit of the operating console 32 includes a data processing device to implement various data processes.

The transmitting/receiving unit 321 includes a transmitting/receiving circuit to transmit and receive the ultrasonic wave within the ultrasonic probe 31, and acquires echo signal by providing transmitting the ultrasonic wave to the subject from the ultrasonic wave vibrators of the ultrasonic probe 31 on the basis of the control signal from the control unit 324, and receiving the ultrasonic wave reflected from the subject with the ultrasonic wave vibrators. For example, the transmitting/receiving unit 321 acquires echo signal by implementing scanning of the subject with the electronic convex scanning system and outputs the echo signal acquired to the slice image generating unit 322. More concretely, the transmitting/receiving unit 321 acquires the echo signal by driving a plurality of ultrasonic vibrators of the ultrasonic wave probe 31 through switching of the positions of vibrators to scan the subject through shifting of the ultrasonic wave beam to the subject and outputs the echo signal to the slice image generating unit 322 by implementing the processes such as amplification, delay, and addition of the echo signal.

The slice image generating unit 322 generates slice images of the slicing planes of the subject on the basis of the echo signal acquired with the ultrasonic probe 31. The slice image generating unit 322 includes a logarithmic amplifier and an envelope detector to detect the envelope after the echo signal outputted from the transmitting/receiving unit 321 is amplified with the logarithmic amplification process. Thereafter, this slice image generating unit 322 calculates intensity of the echo from the respective reflecting points on the sound line by implementing the predetermined data process to such data and thereafter generates the slice image corresponding to the B mode by converting intensity into luminance. Moreover, the slice image generating unit 322 is connected to the storage unit 323 and outputs the slice images generated as explained above to the storage unit 323.

In this embodiment, the slice image generating unit 322 generates the first slice image of the subject as explained above on the basis of the first echo signal acquired by implementing the scanning of the subject with the ultrasonic probe 31 at the first time. Moreover, this slice image generating unit 322 also generates the second slice image on the basis of the second echo signal acquired by implementing the scanning of the subject with the ultrasonic probe 31 at the second time different from the first time. For example, the slice image generating unit 322 generates the slice image of an affected area in the past as the first slice image on the basis of the first echo signal acquired by scanning executed to the affected area of the subject in the past. Moreover, this slice image generating unit 322 also generates a new slice image of the affected area as the second slice image on the basis of the second echo signal acquired by the scanning executed newly to the same affected area of the subject.

The storage unit 323 is constituted, for example, to include a cine-memory and an HDD and stores data of slice images generated with the slice image generating unit 322. The storage unit 323 is connected to the slice image generating unit 322 to temporarily store slice images of a plurality of frames generated with the slice image generating unit 322 to the cine-memory and thereafter stores the slice images to the HDD on the basis of a command from the control unit 324. For example, the storage unit 323 stores slice images of the frames of the two minutes to the cine-memory and thereafter stores these slice images during two minutes to the HDD. In addition, the synthesized images generated with an image synthesizing unit 328 are stored in the HDD. Moreover, the cine-memory of the storage unit 323 is connected to the display unit 41 and the data of slice image of each frame stored in the cine-memory is outputted to the display unit 41. The HDD of the storage unit 323 is also connected to the display unit 41 and the data of the slice image of each frame stored in the HDD is outputted to the display unit 41 on the basis of the command inputted to the operating unit 325 by an operator. Moreover, the synthesized images generated with the image synthesizing unit 328 is then outputted and displayed on the display unit 41.

For example, the control unit 324 includes a computer and programs to control the computer to execute the predetermined data processes which are respectively connected with each unit. The control unit 324 respectively gives the control signals to each unit in order to control the operations.

In this embodiment, the control unit 324 includes, as illustrated in FIG. 1, the first transmitting degree setting unit 324a, the second transmitting degree setting unit 324b, and a convoluting position setting unit 324c.

The first transmitting degree setting unit 324a sets transmitting degree on displaying the first slice image on the display unit 41 through transmission. In this embodiment, the first transmitting degree setting unit 324a sets transmitting degree for displaying the first slice image on the display unit 41 through transmission corresponding to the setting value of the transmitting degree inputted to the first transmitting degree input unit 325a of the operating unit 325 to be explained later. More concretely, the first transmitting degree setting unit 324a sets transmitting degree to display the first slice image in the transmitting degree of 50% on the display unit 41 through transmission when the setting value of transmitting degree of 50% is inputted to the first transmitting degree input unit 325a of the operating unit 325.

The second transmitting degree setting unit 324b sets transmitting degree on displaying the second slice image on the display unit 41 through transmission. In this embodiment, the second transmitting degree setting unit 324b sets transmitting degree on displaying the second slice image on the display unit 41 through transmission corresponding to the setting value of transmitting degree inputted to the second transmitting degree input unit 325b of the operating unit 325 to be explained later. More concretely, the second transmitting degree setting unit 324b sets transmitting degree for displaying the second slice image in the transmitting degree of 50% with the display unit 41 when the setting value of transmitting degree of 50% is inputted to the second transmitting degree input unit 325b of the operating unit 325.

The convolution position setting unit 324c sets position for convoluting the second slice image on the first slice image in the display unit 41. In this embodiment, the convolution position setting unit 324c sets position for convoluting the second slice image on the first slice image in the display unit 41 corresponding to the setting value of the position inputted to the convolution position input unit 325c of the operating unit 325 to be explained later. More concretely, the convolution position setting unit 324c sets position to convolute the second slice image on the first slice image corresponding to the position setting value when the setting value of convoluting position is inputted to the convolution position input unit 325c of the operating unit 325 to be explained later in order to separate the center of the second slice image from the center of the first slice image as much as the predetermined distance.

The operating unit 325 includes an input device, for example, a keyboard, a touch panel, a track ball, a foot switch and a voice input device or the like. The operating unit 325 receives operation information inputted by an operator and outputs operation signal to the control unit 324 in the basis of such operating information.

As illustrated in FIG. 1, the operating unit 325 includes the first transmitting degree input unit 325a, the second transmitting degree input unit 325b, and the convolution position input unit 325c.

The first transmitting degree input unit 325a receives the setting value of transmitting degree inputted by an operator for displaying the first slice image on the display unit 41 through transmission. The first transmitting degree input unit 325a is constituted, for example, to include a keyboard in order to also receive the setting value of transmitting degree when the operator selectively depresses keys of the keyboard.

The second transmitting degree input unit 325b receives the setting value of transmitting degree inputted by the operator for displaying the second slice image on the display unit 41 through transmission. The second transmitting degree input unit 325b is constituted, for example, to include a keyboard and receives the setting value of transmitting degree when the operator selectively depresses keys of the keyboard.

The convolution position input unit 325c receives setting value of location to be inputted by the operator for convoluting the second slice image on the first slice image on the display unit 41. This convolution position input unit 325c is constituted, for example, to include a pointing device and receives the setting value inputted by the operator by selecting the convolution position using the pointing device.

The display unit 41 includes, for example, an LCD device (not illustrated) having a flat display screen and a DSC (Digital Scan Converter) to display images generated by the slice image generating unit 322 and stores the same images in the storage unit 323. The display unit 41 also displays a plurality of slice images stored in the storage unit 323. More concretely, the display unit 41 is connected to the storage unit 323 and converts data of slice image of each frame stored in the cine-memory of the storage unit 323 into the display signal with the DSC and displays such data as the slice image on the display screen of the LCD device on the basis of the command from the control unit 324. Moreover, the display unit 41 is connected to the HDD of storage unit 323 and receives and displays the image data stored in the HDD on the basis of the command inputted to the operating unit 325 from the operator.

In this embodiment, the display unit 41 displays the first slice image and the second display image on the display screen by convoluting the second slice image of the subject generated with the slice image generating unit 322 on the first slice image thereof generated with the slice image generating unit 322. For example, the display unit 41 displays images by convoluting the second slice image as a new slice image of the affected area of the subject to the first slice image as the slice image in the past of the affected area of the same subject.

Here, the display unit 41 displays the first slice image through transmission corresponding to the transmitting degree preset by the first transmitting degree setting unit 324a and also displays the second slice image through transmission corresponding to the transmitting degree preset by the second transmitting degree setting unit 324b. More concretely, when the transmitting degree of 0% is set by the first transmitting degree setting unit 324a, the display unit 41 displays the first slice image in the transmitting degree of 0% through transmission. Moreover, when the second transmitting degree setting unit 324b sets the transmitting degree of 50%, the display unit 41 displays the second slice image in the transmitting degree of 50%.

Moreover, the display unit 41 displays the images by convoluting the second slice image on the first slice image corresponding to the position preset by the convolution position setting unit 324c. More concretely, when the convolution position setting unit 324c sets the convolution position to separate the center of the second slice image from the center of the first slice image as much as the predetermined distance, the images are displayed by separating the center of the second slice image from the center of the first slice image as much as the predetermined distance.

Operations of the ultrasonic diagnostic apparatus 1 in this embodiment of the present invention will be explained below.

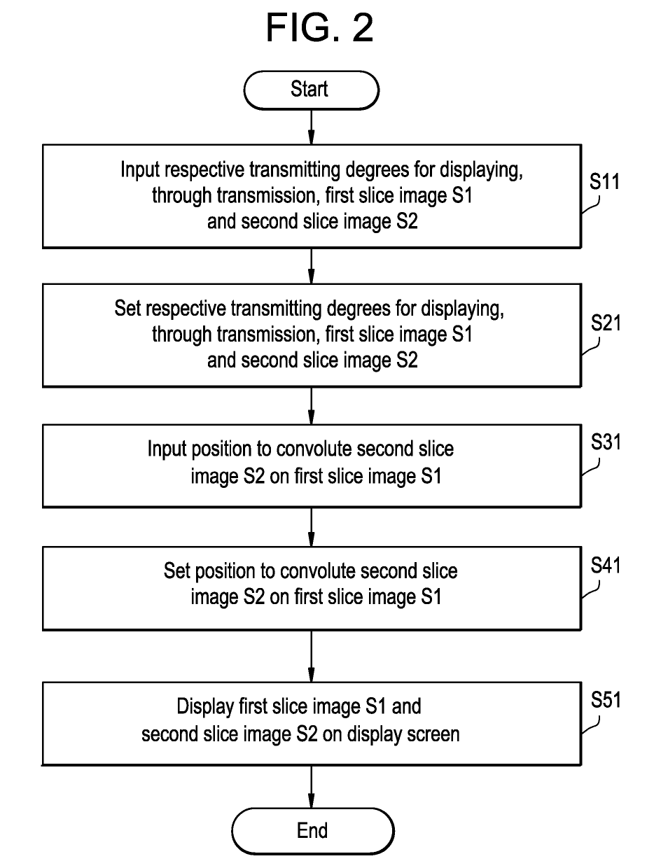

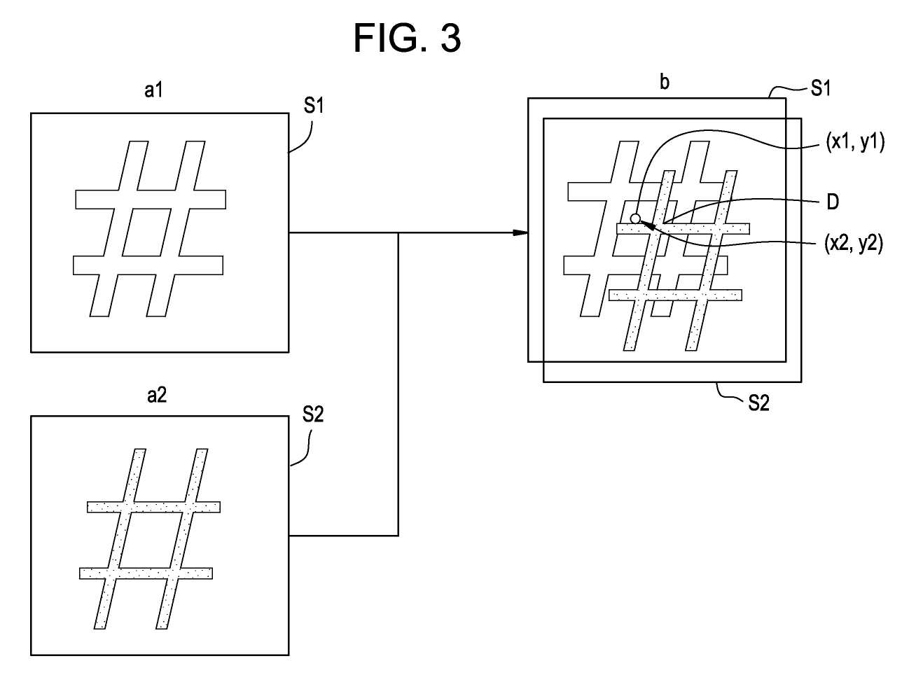

FIG. 2 is a flowchart showing operations of the ultrasonic diagnostic apparatus 1 according to the first embodiment of the present invention. Moreover, FIG. 3 illustrates profiles of operations of the ultrasonic diagnostic apparatus 1 according to the first embodiment. Slice images are simplified in FIG. 3.

As shown in FIG. 2, transmitting degrees for displaying the first slice image S1 and the second slice image S2 through transmission are first inputted (S11).

Here, a setting value of transmitting degree for displaying the first slice image S1 on the display screen of the display unit 41 through transmission is inputted, as illustrated in FIG. 3(a1), to the first transmitting degree input unit 325a by an operator. Next, as illustrated in FIG. 3(a2), a setting value of transmitting degree for displaying the second slice image S2 on the display screen of the display unit 41 through transmission is then inputted to the second transmitting degree input unit 325b by an operator.

In this embodiment, setting values of transmitting degrees for displaying the first slice image S1 of the affected area of the subject generated in the past and the newly generated second slice image S2 of the same affected area of the subject are respectively inputted by an operator. For example, the setting value 0% of transmitting degree is inputted to the first transmitting degree input unit 325a and the setting value 50% of transmitting degree is also inputted thereto by an operator by selectively depressing keys of the keyboard.

Next, as shown in FIG. 2, respective transmitting degrees for displaying the first slice image S1 and the second slice image S2 through transmission are set (S21).

Here, the first transmitting degree setting unit 324a sets a transmitting degree for displaying the first slice image S1 through transmission corresponding to the setting value inputted to the first transmitting degree input unit 325a as explained above. Moreover, the second transmitting degree setting unit 324b sets a transmitting degree for displaying the second slice image S2 through transmission corresponding to the setting value inputted to the second transmitting degree input unit 325b as explained above. For example, the first transmitting degree setting unit 324a sets the transmitting degree to 0% corresponding to the setting value of transmitting degree inputted to the first transmitting degree input unit 325a. Moreover, the second transmitting degree setting unit 324b sets the transmitting degree to 50% corresponding to the setting value of transmitting degree inputted to the second transmitting degree input unit 325b in the same manner.

Next, the position to convolute the second slice image S2 to the first slice image S1 is then inputted as shown in FIG. 2 (S31).

Here, the operator inputs a setting value of position to convolute the second slice image S2 to the first slice image S1 on the display screen of the display unit 41 to the convolution position input unit 325c. More concretely, the operator inputs a setting value of the convolution position to the convolution position input unit 325c by selecting the convolution position using the pointing device. For example, a setting value of the convolution position is inputted to the convolution position input unit 325c by selecting and shifting the second slice image S2 with the pointing device to separate the center of the second slice image S2 from the center of the first slice image S1 as much as the predetermined distance.

Next, the position to convolute the second slice image S2 to the first slice image S1 is set as shown in FIG. 2 (S41).

Here, the convolution position setting unit 324c sets the position to convolute the second slice image S2 to the first slice image S1 on the display screen of the display unit 41 corresponding to the setting value of position inputted to the convolution position input unit 325c explained above. For example, the convolution position setting unit 334c sets the position to convolute the second slice image S2 to the first slice image S1 in order to separate the center of the second slice image S2 from the center of the first slice image S1 as much as the predetermined distance on the basis of the setting value of position inputted to the convolution position setting unit 325c explained above.

Next, the first slice image S1 and the second slice image S2 are displayed on the display screen as shown in FIG. 2 (S51).

Here, the display unit 41 displays the first slice image S1 and the second slice image S2 on the display screen thereof through convolution. For example, the slice images are displayed by convoluting the newly generated second slice image S2 of the affected area of the subject to the first slice image S1 of the same affected area generated in the past.

More concretely, the display unit 41 respectively displays the first slice image S1 and the second slice image S2 through convolution respectively corresponding to the transmitting degree preset by the first transmitting degree setting unit 324a and the transmitting degree preset by the second transmitting degree setting unit 324b. For example, the first slice image is displayed in the transmitting degree of 0% through transmission, while the second slice image is displayed in the transmitting degree of 50% through transmission. Namely, display is conducted in the manner that the pixel value of the second image S2 becomes 50% and the pixel value of the first slice image S1 becomes 50% through transmission for the pixels positioned through convolution behind the second slice image S2 on the display screen. Namely, display is conducted with the pixel value attained by adding the pixel value of 50% in the first slice image S1 and the pixel value of 50% in the second slice image S2 for the pixels where the first slice image and the second slice image S2 are convoluted on the display screen.

Moreover, the display unit 41 displays images by convoluting the second slice image S2 on the first slice image S1 corresponding to the position preset by the convolution position setting unit 324c as illustrated in FIG. 3(b). For example, display is conducted in the manner that the center coordinate (x2, y2) of the second slice image S2 is separated as much as the predetermined distance D from the center coordinate (x1, y1) of the first slice image S1. Namely, the images are displayed by convoluting the second slice image S2 on the first slice image S1 in the manner that the center coordinate (x2, y2) of the second slice image S2 is different from the center coordinate (x1, y1) of the first slice image S1.

As explained above, according to this embodiment, the display unit 41 displays the first slice image S1 through transmission corresponding to the transmitting degree preset by the first transmitting degree setting unit 324a and also displays the second slice image S2 through transmission corresponding to the transmitting degree preset by the second transmitting degree setting unit 324b. Moreover, the display unit 41 displays images by convoluting the second slice image S2 on the first slice image S1 corresponding to the position preset by the convolution position setting unit 324c. For example, the display unit 41 displays images through transmission by convoluting with each other the first slice image S1 of the affected area of the subject generated in the past and the newly generated second slice image S2 of the same affected area of the subject.

Therefore, in this embodiment, the portions to be compared can be displayed adjacently with each other on the display screen in view of comparing a plurality of slice images S1 and S2. Accordingly, the portions to be compared can be detected easily and can also be diagnosed quickly. Thereby, this embodiment can realize higher diagnosis efficiency.

In this embodiment explained above, the ultrasonic diagnostic apparatus 1 corresponds to the image diagnostic apparatus of the present invention. In this embodiment, the ultrasonic probe 31 corresponds to the scanning unit of the present invention. In this embodiment, moreover, the display unit 41 corresponds to the display unit of the present invention. Moreover, in this embodiment, the slice image generating unit 322 corresponds to the slice image generating unit of the present invention. In addition, the first transmitting degree setting unit 324a corresponds to the first transmitting degree setting unit of the present invention. Moreover, in this embodiment, the second transmitting degree setting unit 324b corresponds to the second transmitting degree setting unit of the present invention. Moreover, in this embodiment, the convolution position setting unit 324c corresponds to the convolution position setting unit of the present invention. Moreover, in this embodiment, the first transmitting degree input unit 325a corresponds to the first transmitting degree input unit of the present invention. Moreover, in this embodiment, the second transmitting degree input unit 325b corresponds to the second transmitting degree input unit of the present invention. Moreover, in this embodiment, the convolution position input unit 325c corresponds to the convolution position input unit of the present invention. Moreover, in this embodiment, the first slice image S1 corresponds to the first image and the first slice image of the present invention. Moreover, in this embodiment, the second slice image S2 corresponds to the second image and the second slice image of the present invention.

### Second Embodiment

The second embodiment of the present invention will be explained below.

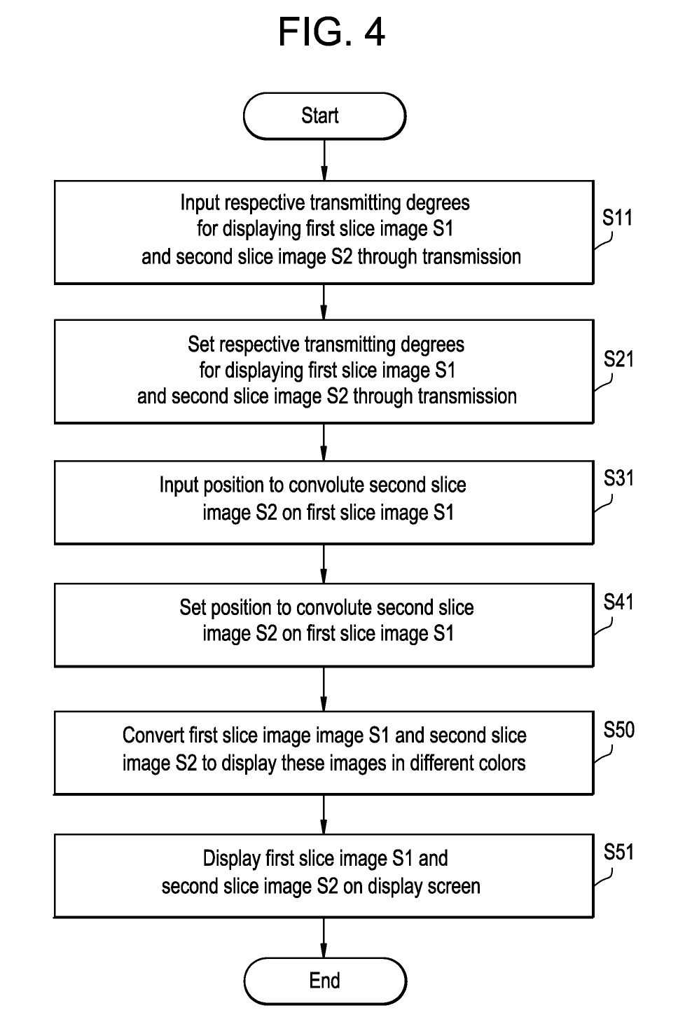

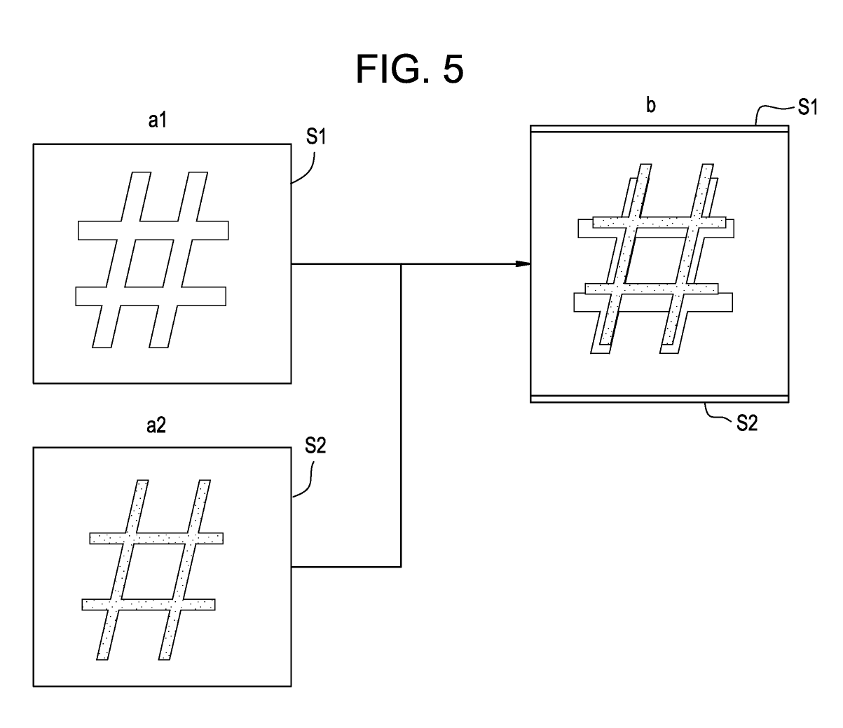

FIG. 4 is a flowchart showing operations of the ultrasonic diagnostic apparatus 1 according to the second embodiment of the present invention. Moreover, FIG. 5 illustrates profiles of operations of the ultrasonic diagnostic apparatus 1 in the second embodiment of the present invention.

This second embodiment is different in operations of the ultrasonic diagnostic apparatus 1 from the first embodiment 1. The second embodiment is similar to the first embodiment, except for such difference in the operations of the ultrasonic diagnostic apparatus 1. The common contents through the first and second embodiments are eliminated in explanation.

Like the first embodiment, after respective transmitting degrees for displaying the first slice image S1 and the second slice image S2 through transmission are inputted (S11) as illustrated in FIG. 4, respective transmitting degrees for displaying the first slice image S1 and the second slice image S2 through transmission are set (S21).

Here, as illustrated in FIG. 5(a1), an operator inputs a setting value of transmitting degree for displaying the first slice image S1 through transmission on the display screen of the display unit 41 to the first transmitting degree input unit 325a like the first embodiment and this setting value is then set to the first transmitting degree setting unit 324a. Moreover, as illustrated in FIG. 5(a2), the operator inputs a setting value of transmitting degree for displaying the second slice image S2 through transmission on the display screen of the display unit 41 to the second transmitting degree input nit 325b and this setting value is also set to the second transmitting degree setting unit 324b.

Like the first embodiment, after position to convolute the second slice image S2 on the first slice image S1 is inputted (S31), position to convolute the second slice image S2 on the first slice image S1 is set (S41).

In this second embodiment, the convolution position setting unit 324c sets the position to convolute the second slice image S2 on the first slice image S1, in the manner that the center position of the second slice image S2 is not separated from the center position of the first slice image S1 as much as the predetermined distance and the center position of the second slice image S2 is located at the center position of the first slice image S1, on the basis of the setting value of the position inputted to the convolution position input unit 325c.

Next, as shown in FIG. 4, the first slice image S1 and the second slice image S2 are converted to show different colors from each other (S50).

Here, the display unit 41 converts the first slice image S1 and the second slice image S2 to show different colors from each other.

More concretely, the display unit 41 converts at least one pixel data of the first slice image S1 and the second slice image S2 to show difference at least in one element of hue, luminosity, saturation between the first slice image S1 and the second slice image S2. For example, conversion is conducted so that the first slice image S1 is shown in black and the second slice image S2 is shown in blue in order to provide different hues between the first slice image S1 and the second slice image S2.

Next, like the first embodiment, the first slice image S1 and the second slice image S2 are displayed on the display screen as shown in FIG. 4 (S51).

Here, like the first embodiment, the display unit 41 displays respectively the first slice image S1 and the second slice image S2 through transmission corresponding respectively to the transmitting degree preset by the first transmitting degree setting unit 324a and to the transmitting degree preset by the second transmitting degree setting unit 324b. In this case, as illustrated in FIG. 5(b), the display unit 41 displays the first slice image S1 and the second slice image S2 in colors different from each other. For example, the first slice image S1 is displayed in black and the second slice image S2 in blue.

Moreover, as illustrated in FIG. 5(b), the display unit 41 displays images in the manner that the second slice image S2 is convoluted on the first slice image S1 corresponding to the position preset by the convolution position setting unit 324c. For example, the images are displayed in the manner that the center position of the second slice image S2 is located at the center position of the first slice image S1.

In this second embodiment, as explained above, the display unit 41 displays, through transmission, the images by convoluting each other the first slice image S1 of the affected area of the subject generated in the past and the newly generated second slice image S2 of the same affected area of the subject as in the case of the first embodiment. Here, the first slice image S1 and the second slice image S2 are displayed in colors different from each other.

Accordingly, in this second embodiment, the portions to be compared can be identified easily in the case where a plurality of slice images are compared and thereby diagnostic process can be realized quickly. Therefore, higher diagnosis efficiency can be realized according to the second embodiment.

### Third Embodiment

The third embodiment of the present invention will be explained below.

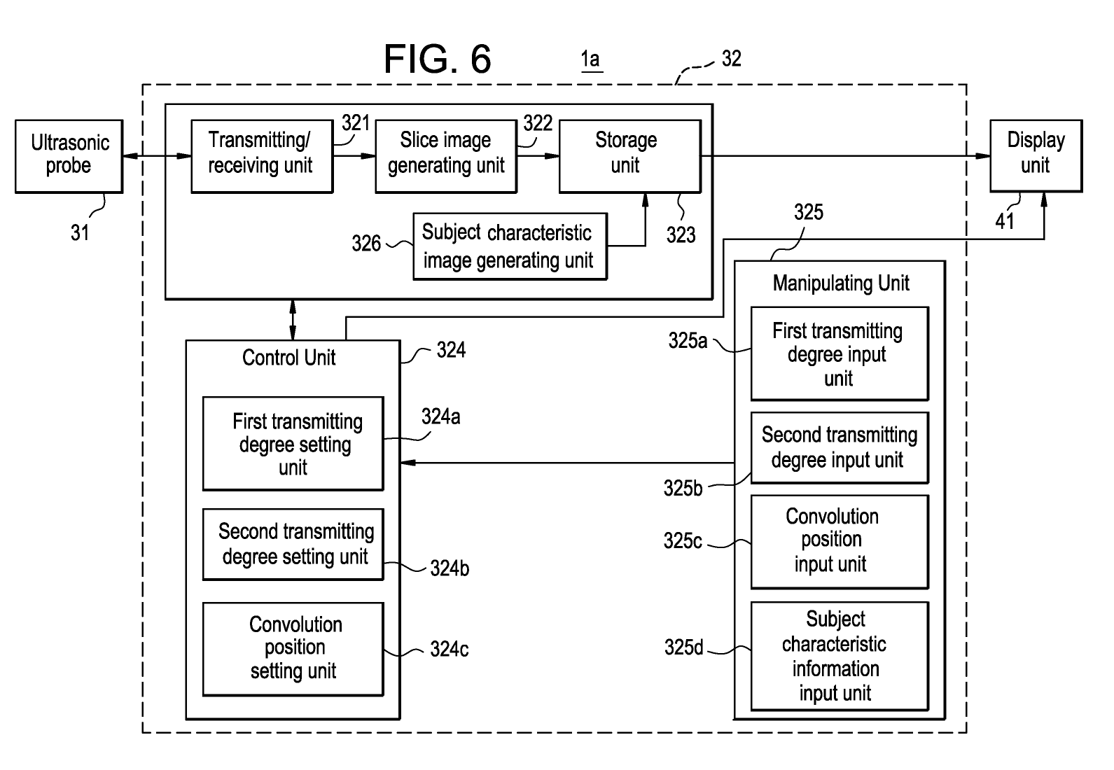

FIG. 6 is a block diagram illustrating a structure of an ultrasonic diagnostic apparatus 1a according to the third embodiment of the present invention.

As illustrated in FIG. 6, the diagnostic apparatus 1a of this third embodiment allows, unlike the first embodiment, provision of a subject characteristic image generating unit 326 to the operating console 32. Moreover, a subject characteristic information input unit 325d is provided to the operating unit 325 of the operating console 32. This embodiment is similar to the first embodiment, except for this structure. Therefore, the identical portion is not explained here.

An operator inputs characteristic information regarding characteristic of a subject to the subject characteristic information input unit 325d. This subject characteristic information input unit 325d is constituted, for example, to include a keyboard. The characteristic information of the relevant subject can be inputted when the operator selectively depresses the keys on the keyboard. For example, the characteristic information showing characteristic of the subject such as name, sex, and diagnostic report or the like can be inputted.

The subject characteristic information generating unit 326 generates a subject characteristic image showing characteristic of the subject on the basis of the characteristic information inputted to the subject characteristic information input unit 325d. For example, the subject characteristic image is generated to show with letters characteristic of the subject such as name and sex or the like. The subject characteristic image generating unit 326 is connected to a storage unit 323 to output the subject characteristic image generated to the storage unit 323.

In this third embodiment, the display unit 41 receives, from the storage unit 323, the subject characteristic image data generated by the subject characteristic image generating unit 326 and then displays the same data on the display screen thereof. For example, the display unit 41 displays the subject characteristic image in the manner to convolute the same image on the slice image generated with the slice image generating unit 322.

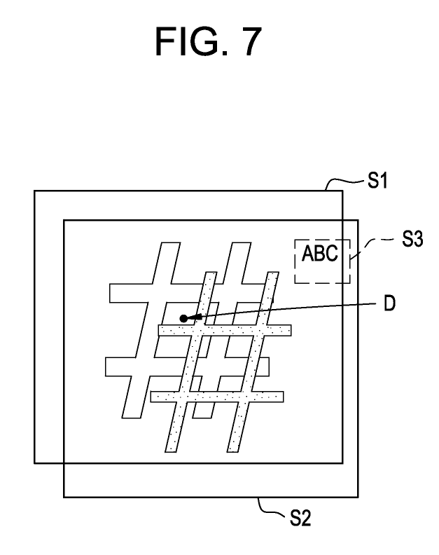

FIG. 7 illustrates an image displayed on the display unit 41 in the third embodiment of the present invention.

As illustrated in FIG. 7, the display unit 41 displays, like the first embodiment, the subject characteristic image S3 generated as explained above on the display screen to convolute on the first slice image S1 of the affected area of the subject generated in the past and on the newly generated second slice image S2 of the same affected area of the subject. Namely, the display unit 41 displays, like the first embodiment, the first slice image S1 and the second slice image S2 corresponding to the preset transmitting degree and to the convolution position and also displays the subject characteristic image S3 corresponding to the preset transmitting degree and the convolution position. For example, the subject characteristic image S3 is displayed through convolution on the first slice image S1 to displace the center coordinate (x3, y3) of the subject characteristic image S3 from the center position (x1, y1) of the first slice image S1.

As explained above, in this third embodiment, the display unit 41 displays through transmission, like the first embodiment, the slice images S1 and S2 by convoluting with each other the first slice image S1 of the affected area of the subject generated in the past and the newly generated slice image S2 of the same affected area. Moreover, the display unit 41 also displays, in the display screen thereof, the subject characteristic image S3 by convoluting the same image S3 on the first slice image S1 and the second slice image S2.

As explained above, in this third embodiment, since the subject characteristic image S3 is convoluted, for display on the display screen, on the first slice image S1 and the second slice image S2, the first slice image S1 and the second slice image S2 can be displayed in large size and characteristic of the subject can be detected easily from the subject characteristic image S3. Accordingly, this third embodiment is capable of executing the diagnostic process quickly by easily detecting characteristic of the subject. Therefore, this third embodiment can realize higher diagnosis efficiency.

In this embodiment, the subject characteristic information input unit 325d corresponds to the subject characteristic information input unit of the present invention. Moreover, in this third embodiment, the subject characteristic image generating unit 326 corresponds to the subject characteristic image generating unit of the present invention. Moreover, in this third embodiment, the subject characteristic image S3 corresponds to the second image and the subject characteristic image of the present invention. Moreover, in this third embodiment, the first slice image S1 and the second slice image S2 respectively correspond to the first image of the present invention.

Moreover, the present invention is not limited only to the embodiments explained above for embodying the present invention and the present invention allows various changes and modifications.

In the embodiments explained above, images are displayed through transmission by convoluting with each other the first slice image S1 of the affected area of the subject generated in the past and the newly generated second slice image S2 of the same affected area. However, the present invention is not limited thereto. For example, the ultrasonic probe 31 acquires the third echo signal by implementing the scanning to the first subject as the subject, the slice image generating unit 322 generates the third slice image of the first subject on the basis of the third echo signal after the ultrasonic probe 31 acquires the fourth echo signal by implementing the scanning to the second subject different from the first subject, and when the fourth slice image of the second subject is generated on the basis of the fourth echo signal, the display unit 41 may display the images by convoluting the fourth slice image of the second subject on the third slice image of the first subject. More concretely, the present invention can also be applied to the case where the first slice image picked up for the imaging region corresponding to the affected area of the first subject and the slice image picked up for the imaging region corresponding to the affected area of the second subject different from the first subject are displayed on the display screen thereof through convolution with each other.

Moreover, in the embodiments explained above, the ultrasonic diagnostic apparatus is used as the image diagnostic apparatus, but the present invention is not limited thereto. For example, the present invention can also be applied to the image diagnostic apparatus which includes a scanning unit to acquire the projected data, as the raw data, obtained through the scanning by irradiating the subject with the radioactive ray and then detecting the radioactive ray having transmitted through the subject and then reconstitutes the slice images of the subject on the basis of such projected data. Namely, the present invention may be applied, for example, to a radioactive ray photographing apparatus such as an X-ray CT apparatus.

In addition, the present invention may also be applied to an image diagnostic apparatus such as a magnetic resonance imaging apparatus which includes a scanning unit to acquire the magnetic resonance signal, as the raw data, generated in the subject by transmitting the RF pulse to the same subject within the magnetostatic field space and constitutes slice images of the subject on the basis of the magnetic resonance signal thereof.

In the embodiments explained above, display of images by convoluting with each other two slice images and display of images by convoluting one characteristic image to such two slice images have been explained but the present invention is not limited thereto. For example, the present invention can also be applied for the display of desired number of images where three or more slice images are convoluted with each other or two or more subject characteristic images are convoluted with each other.

Moreover, in the embodiments explained above, when the images are displayed on the display screen by convoluting the second slice image of the subject on the first slice image of the same subject, the display unit is capable of displaying the second slice image by shifting the same image little by little to the first slice image on the basis of an instruction from an operator. With such display of images through gradual shifting of the one image, different point can be confirmed easily.

Moreover, when the images are displayed on the display screen by convoluting the second slice image of the subject on the first slice image of the same subject in the embodiments explained above, the display unit may display the images in the manner that the second image can be rolled up from the first image. Namely, the images can also be displayed in the manner that the edge of the second image is rolled up from the first image.

Many widely different embodiments of the present invention may be configured without departing from the spirit and scope of the present invention. It should be understood that the present invention is not limited to the specific embodiments described in the specification, except as defined in the appended claims.

1. An image diagnostic apparatus including a display device for displaying images on a display screen thereof by convoluting a second image of a subject on a first image of the subject, comprising:
a first transmitting degree setting device for setting a transmitting degree upon displaying the first image on the display device through transmission; and
a second transmitting degree setting device for setting a transmitting degree upon displaying the second image on the display device through transmission,
wherein the display device displays the first image through transmission corresponding to the transmitting degree set by the first transmitting degree setting device and displays the second image through transmission corresponding to the transmitting degree set by the second transmitting degree setting device.

2. The image diagnostic apparatus according to claim 1, comprising:
a first transmitting degree input device for inputting a setting value of a transmitting degree by an operator upon displaying the first image on the display device through transmission; and
a second transmitting degree input device for inputting a setting value of a transmitting degree by an operator upon displaying the second image on the display device though transmission,
wherein the first transmitting degree setting device is capable of setting a transmitting degree upon displaying the first image on the display device through transmission corresponding to the setting value of the transmitting degree inputted to the first transmitting degree input device, and
wherein the second transmitting degree setting device is capable of setting a transmitting degree upon displaying the second image on the display device through transmission corresponding to the setting value of the transmitting degree inputted to the second transmitting degree input device.

3. The image diagnostic apparatus according to claim 1, comprising:
a convolution position setting device for setting a convolution position of the second image on the first image in the display device,
wherein the display device displays the second image on the first image through convolution corresponding to the position preset by the convolution position setting device.

4. The image diagnostic apparatus according to claim 3, comprising:
a convolution position inputting device for inputting, by an operator, a setting value of a convolution position of the second image on the first image on the display device,
wherein the convolution position setting device is capable of setting a convolution position of the second image on the first image on the display device corresponding to the setting value of the position inputted to the convolution position inputting device.

5. The image diagnostic apparatus according to claim 1, comprising:
a scanning device for obtaining raw data by scanning the subject; and
a slice image generating device for generating slice images of the subject on the basis of the raw data obtained by the scanning device,
wherein the display device displays a slice image generated by the slice image generating device as at least one of the first image and the second image.

6. The image diagnostic apparatus according to claim 5, wherein the scanning device acquires a first raw data by scanning the subject at a first time and acquires a second raw data by scanning the subject at a second time different from the first time,
wherein the slice image generating device generates a first slice image of the subject on the basis of the first raw data and generates a second slice image of the subject on the basis of the second raw data, and
wherein the display device displays the first slice image as the first image and displays the second slice image as the second image.

7. The image diagnostic apparatus according to claim 5, wherein the scanning device acquires a third raw data by scanning a first subject as the subject and acquires a fourth raw data by scanning a second subject different from the first subject as the subject,
wherein the slice image generating device generates a third slice image of the first subject on the basis of the third raw data and generates a fourth slice image of the second subject on the basis of the fourth raw data, and
wherein the display device displays the third slice image as the first image and displays the fourth slice image as the second image.

8. The image diagnostic apparatus according to claim 5, wherein the scanning device acquires an echo signal, as the raw data, obtained by transmitting ultrasonic wave to the subject and implementing scanning to receive the ultrasonic wave reflected from the subject to which the ultrasonic wave has been transmitted.

9. The image diagnostic apparatus according to claim 5, wherein the scanning device acquires, as the raw data, a projected data obtained by scanning the subject to detect the radioactive ray transmitted through the subject.

10. The image diagnostic apparatus according to claim 5, wherein the scanning device transmits a RF pulse to the subject in magnetostatic field and acquires, as the raw data, magnetic resonance signal generated in the subject.

11. The image diagnostic apparatus according to claim 1, comprising:
a subject characteristic information input device inputted by an operator, for inputting characteristic information regarding characteristic of the subject; and
a subject characteristic image generating device for generating subject characteristic image showing characteristic of the subject on the basis of the characteristic information inputted to the subject characteristic information input device,
wherein the display device displays the subject characteristic image generated by the subject characteristic image generating device as at least one of the first image and the second image.

12. The image diagnostic apparatus according to claim 1, wherein the display device displays the first image and the second image after these are converted to show different colors from each other.
